(19) United States
(12) Patent Application Publication
(10) Pub. No.: US 2026/0183007 A1
(43) Pub. Date: Jul. 2, 2026

(54) CATHETER HAVING A FUNCTIONAL ELEMENT

(71) Applicant: Straub Medical AG, Wangs (CH)

(72) Inventor: Bruno Bahnmueller, Wangs (CH)

(21) Appl. No.: 19/129,904

(22) PCT Filed: Nov. 17, 2022
(86) PCT No.: PCT/EP2022/082250

§ 371 Date: May 14, 2025

(51) Int. Cl. A61B 17/221; A61B 17/00; A61B 17/22

(52) U.S. Cl. CPC A61B 17/221; A61B 2017/00367; A61B 2017/00477; A61B 2017/22038; A61B 2017/2215

(57) ABSTRACT

Catheter (1) having a functional element (4) having at least a deployed configuration and an elongated configuration, the functional element (4) comprising filaments (5) and first (6a) and second (6b) connectors, the first connector (6a) connected to a first end (5a) of the filaments, and the second connector (6b) connected to a second end (5b) of the filaments, wherein in the elongated configuration, the first connector (6a) is at a distal end (d-e) of the functional element and the second connector (6b) is at a proximal end (p) of the functional element, and in the deployed configuration, the functional element is configured to act as a filter penetrable to fluid, the filaments forming turn-around points (5c) forming a distal end (d-d) of the filter, wherein the first connector (6a) is proximal to the turn-around points (5c), the catheter further comprising an actuator (7) configured to move the first connector (6a) proximally and to thereby facilitate transformation of the functional element (4) from the elongated configuration into the deployed configuration of the functional element.

### TECHNICAL FIELD

The present disclosure relates to catheters having a functional element, catheter systems comprising such catheter, and methods of using a functional element. Also, the use of a catheter having a functional element is contemplated.

### BACKGROUND

Catheters may have a filter to avoid blood clot particles being carried away, as emboli, by the blood flow to undesired locations within the body. Such emboli may travel to organs and cause an embolization.

A vascular filter may be used to trap such emboli. Conventional filters may have sharp edges, or hooks for attaching the filter to the inner wall of the vessel.

It is desired to provide an atraumatic filter and to reduce damage and trauma to the vessel wall.

### SUMMARY

According to the disclosure, a catheter has a functional element comprising filaments and at least a deployed configuration and an elongated configuration. The functional element comprises first and second connectors, the first connector connected to a first end of the filaments, and the second connector connected to a second end of the filaments. In the elongated configuration, the first connector is at a distal end of the functional element and the second connector is at a proximal end of the functional element. In the deployed configuration, the functional element is configured to act as a filter penetrable to fluid, the filaments forming turnaround points forming a distal end of the filter, wherein, in the deployed configuration, the first connector (and also the second connector) is proximal to the turnaround points. The first connector in the deployed configuration may be proximal to the location of the distal end of the functional element when the functional element is in the elongated and/or deployed configuration. The location of the first connector in the deployed configuration may be proximal to the location of the first connector in the elongated configuration. The catheter further comprises an actuator configured to move the first connector proximally and to thereby facilitate transformation of the functional element from the elongated configuration into the deployed configuration.

By way of the turnaround points formed by the filaments, damage to the bodily wall upon contact between the turnaround points and the inner surface of the wall can be avoided. The turnaround points may represent returning loops and/or may be regarded as folded back filaments. The turnaround points are smooth and have no sharp tips. The turnaround points are atraumatic. The functional element (filter) of the disclosure may gently engage bodily walls.

The elongated configuration may refer to the configuration in which the filaments connecting the first and second connectors are substantially straight. This may be a low-profile configuration. The elongation may refer to the maximum extension of the filaments in the longitudinal direction.

The filter is penetrable to fluid, in particular to blood flow. The filter is not penetrable to, but captures particles, such as emboli, e.g. blood clot particles. The functional element is transformable from the elongated configuration to the deployed configuration by moving the first connector in the proximal direction, i.e. proximally.

The functional element/catheter of the disclosure is configured to capture emboli transported with the blood flow into the proximal direction.

The functional element is configured to act or serve as a filter at least in the deployed configuration. However, it is not excluded that the functional element may also act as a filter when in the elongated configuration.

The functional element (filter) is versatile in that the position of the turnaround points is adjustable. For instance, the longitudinal position of the turnaround points may be determined by the position of the first connector in the deployed configuration. The further the first connector is moved proximally, the more proximal the turnaround points are located, i.e. the shorter the filter is in the longitudinal direction.

The radial distance of the turnaround point relative to each other may depend on the diameter of the bodily wall, in particular if the filter is self-expanding. In case of a relatively small bodily lumen, the umbrella formed by the filaments does not open that much, so that the radial extension of the filter may be smaller. If the bodily lumen is larger, the umbrella opens further.

Optionally, the angular distribution of the filaments and, accordingly, of the turnaround points is equal relative to each other. For example, about 16 to 64, optionally 48, filaments may be arranged at the connectors, at relatively equal angular distribution, for example.

Optionally, in the deployed configuration, the first connector is located proximal to the second connector.

If the actuator is pulled further proximally when the functional element is in the deployed configuration, the actuator may facilitate transformation of the functional element from the deployed configuration to a retracted configuration. Some retracted configurations may be regarded as an inversion of the elongated configuration in that the order of the first and second connectors along the proximal/distal direction may be inverted.

When the filter has captured emboli in the deployed configuration, the emboli are at the distal side of the filter. If the distal side is, during retraction, moved proximally, such movement may ensure reliable transport of the emboli. Thus, it may be avoided that captured emboli escape from the filter during retraction.

Optionally, the actuator comprises a pull wire configured to pull the first connector in the proximal direction. If a pull wire is used to pull the first connector in the proximal direction, efficient and reliable realization may be provided. The pull wire may be connected (directly or indirectly) to the first connector.

Optionally, the second connector has a through hole configured for the first connector to pass through. Optionally, the second connector may be ring-shaped. The ring may represent a through-hole through which the first connector may pass. Such an embodiment may allow for swift “inversion” of the order of the first and second connectors along the proximal/distal direction. Also, such a configuration (or another one) may allow the second connector and the first connector to be fixed relative to each other. As such, the first and second connectors may fix themselves relative to each other.

Optionally, the first connector is smaller than the through-hole of the second connector.

Optionally, the first connector has a diameter that is smaller than a diameter of the second connector. In this way, the first connector may be pulled through the second connector. For example, the first connector may be shaped as an oval ring. As long as the semi-minor axis is less than a diameter of the second connector, the first connector can be pulled through the second connector. If the semi-major axis of the first connector is greater than a diameter of the second connector, but the semi-minor axis diameter of the first connector is smaller than a diameter of the second connector, then the first connector may pass through the second connector and “lock” in place.

Optionally, the first connector is shaped such that it can pass through an opening of the second connector when pulled in a proximal direction. Preferably, the first connector is shaped such that once through the opening of the second connector, the first connector does not return to a location distal of the second connector.

Optionally, the first and second connectors have an opening to allow a guidewire to pass through.

Optionally, the first connector has a central opening for receiving a guidewire and/or is ring-shaped. In some embodiments, the first connector may allow for the use of a guidewire which extends through the center of the first connector. The ring-shape may support that the first connector is deformable. This may support the self-fixing of the first and second connectors as described above.

Optionally, the filaments may be wires and/or fibers. The filaments form a mesh and/or net structure. The filaments may comprise a stent-like wire net structure. As such, the filter may be self-expandable. The filaments may be made of Nitinol. Alternatively or additionally, the filaments may include loops.

A length of the filaments between the first and second connectors in the elongated configuration may be between 20 and 100 mm, optionally between 40 to 60 mm.

Optionally, in the deployed configuration, the filaments form an umbrella shape, wherein the open side of the umbrella faces in the distal direction. As such, the filaments may substantially form a half sphere, wherein the opening of the umbrella points distally. The umbrella may be regarded as a half-sphere.

Optionally, in the deployed configuration, the first and second connectors may be located in proximity. One way to locate the first and second connectors in proximity to each other may be positioning the first connector proximal to the turnaround points in the deployed configuration.

Alternatively or additionally, the first connector may be connected to the second connector. For example, a clip or barb may be provided at the first connector, so as to facilitate engagement with the second connector. Also the ring-shape of the first and second connectors, as described above and below, may provide the desired (self-)connection.

Optionally, the first connector may comprise compliant (flexible) material such that the first connector is deformable. In particular, the first connector may be configured to be deformed to pass through the second connector when moved proximally by the actuator and configured to resume its original shape when the first connector is proximal to the second connector. In such embodiment, the first connector and the second connector are provided such that they represent an efficient and simple realization for a first connector configured to pass through a second connector, wherein the first connector is connected to the second connector once the first connector has passed through the second connector. Specifically, if the first and second connectors are ring-shaped, deformation of the first connector and resumption of its original shape may be facilitated.

Optionally, in the elongated configuration, a virtual outer shell of the filaments may at least partially exhibit a tubular shape. More specifically, at least a part of the section of the filaments between the first and second connectors, optionally the central region between the first and second connectors, is substantially tubular. This may allow for expedient installation/transport to the deployment area prior to deployment.

Optionally, in the deployed configuration, the turnaround points are configured to conform to a blood vessel.

Optionally, in the deployed configuration, the second connector is arranged at a distal end of a tube of the catheter or is arranged distal to the distal end of a tube of the catheter.

Optionally, in a deployed configuration, the filter is configured to retain particles/emboli having a size of at least 1.0 or 1.2 mm. The configuration of the filter can vary in a deployed configuration depending on the size of the emboli sought to be captured. In other embodiments, the filter, in the deployed configuration, is configured to retain particles having a size of at least 1.5 mm.

The present disclosure is also directed to a catheter system comprising, in addition to the catheter as described, a guide wire, a hemostatic valve, and a pull wire, and a sheath.

Also, the present disclosure relates to a method of retaining emboli.

Generally, the distal direction is defined as the direction pointing away from the user (practitioner/surgeon). The proximal direction extends opposite to the distal direction and is the direction pointing away from the patient and towards the user (practitioner/surgeon).

### BRIEF DESCRIPTION OF THE DRAWINGS

FIG. 1 depicts a schematic cross-section of a catheter of the disclosure in the elongated configuration.

FIGS. 2a and 2b depict a schematic view of a functional element of the present disclosure in the deployed configuration.

FIG. 3 depicts a schematic view of a catheter of the present disclosure in the deployed configuration of the functional element.

### DETAILED DESCRIPTION

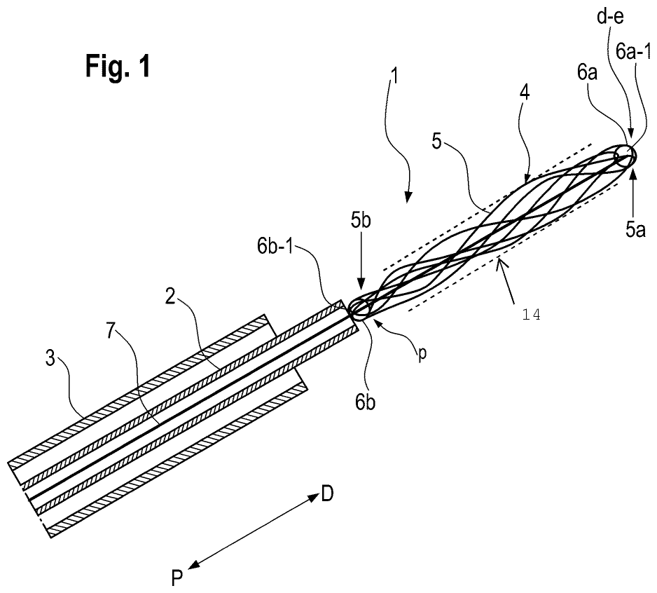

A catheter 1 having a functional element (filter) 4 is shown in FIG. 1. FIG. 1 shows an elongated configuration, which the functional element 4 may have during insertion, i.e. when the functional element 4 is, together with the remaining catheter 1, introduced into a patient (not shown).

The catheter 1 comprises a catheter tube 2. The catheter tube 2 may be at least partially received within a sheath 3. The functional element 4 is provided at a distal end of the catheter tube 2. Put differently, the functional element 4 is provided at a distal end of the catheter 1.

The distal direction D points to the right side of FIG. 1, whilst the proximal direction P points to the left side in FIG. 1. These directions may define longitudinal positions.

The functional element 4 comprises a first 6a and a second connector 6b. The first connector 6a is connected to a first, distal end 5a of the filaments, and the second connector 6b is connected to a second, proximal end 5b of the filaments. The functional element 4 comprises filaments 5.

In the elongated configuration as shown in FIG. 1, the first connector 6a is at a distal end d-e of the functional element 4. The second connector 6b is at a proximal end p of the functional element 4. The elongated configuration may be regarded as a folded configuration of the functional element 4.

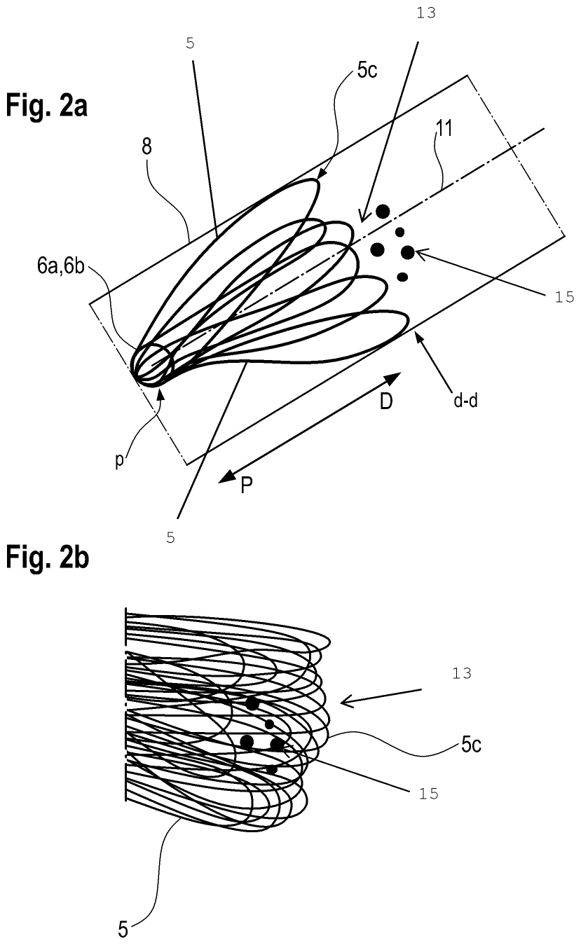

The catheter 1 as shown in FIG. 1 comprises an actuator 7. The actuator 7 allows the first connector 6a to move proximally and to thereby facilitate transformation of the functional element 4 from the elongated configuration into the deployed configuration, which is shown in FIGS. 2a and 2b.

The actuator 7 may comprise a pull wire configured to pull the first connector 6a in the proximal direction P. As such, the actuator 7 may facilitate retraction of the first connector 6a.

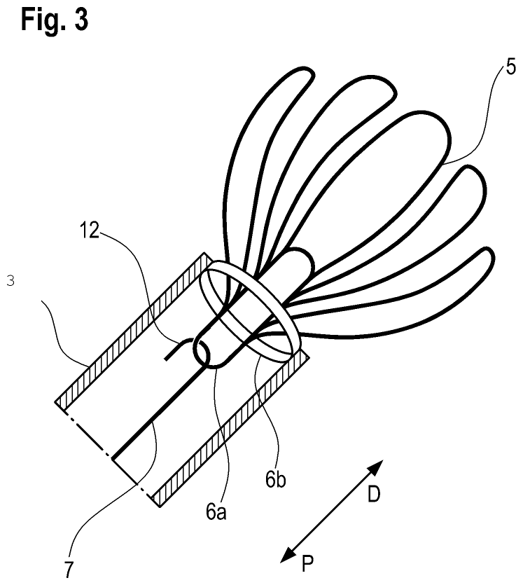

In FIG. 3, the hook 12 of the pull wire 7 engages the first connector 6a. By retracting the pull wire 7 when the filter 4 is in the deployed configuration, the functional element (filter) 4 is withdrawn proximally, together with the emboli 15 (see FIG. 2b) captured in the filter 4.

It is noted that in the present description, the actuator 7 is used twice, i.e. for transformation of the filter 4 from the elongated to the deployed configuration, and for retraction of the filter 4 for removal of the filter 4 from the vasculature. However, different actuators 7, such as different pull wires, may be used. For the sake of hygiene, pull wires 7 may be single use/disposable entities. Hence, when referring to “the” actuator 7, it may mean that two different actuators are encompassed.

As indicated in FIG. 1, the second connector 6b may have a through hole 6b-1 and may be ring-shaped. The first connector 6a may have a central opening 6a-1 and may also be ring-shaped.

FIG. 1 shows that the filaments 5 form a mesh, such as a stent-like structure.

In the elongated configuration shown in FIG. 1, the filaments 5 in their entirety exhibit at least in part a tubular shape, defined by a virtual outer shell 14 (see dotted lines) being tubular. In such a configuration, the filaments 5 extend between the first and second connectors 6a, 6b without turnaround points between the first and second connectors 6a, 6b. In other words, the filaments 5 are located both distally of the second connector and proximally of the first connector, in the elongated configuration.

In the deployed configuration as shown in FIGS. 2a and 2b, the functional element 4 corresponds to a filter and has turnaround points 5c formed by the filaments 5. The turnaround points 5c form a distal end d-d of the filter 4. As can be taken from FIG. 2a, the first connector 6a is proximal to the turnaround points 5c and is proximal to the location of distal end d-e of the functional element 4 when the functional element is in the elongated configuration (shown in FIG. 1).

After deployment, the filter 4 may remain in the deployed state in the vessel 8, as long as filtering is desired. Once the filter 4 has been deployed, the catheter tube 2 and the sheath 3 may be retracted and removed from the vessel 8. The filter 4 remains in the vessel alone. Therefore, it is preferable that the filter 4 is configured to disengage from the remaining entities, such as the tube 2, the sheath 3 and the pull wire 7.

In the deployed configuration shown in FIGS. 2a and 2b, the filaments 5 and filter 4 may be regarded as having an umbrella-like shape. Such configuration may also be regarded as a half sphere. The open side, i.e. a distal opening 13, of the umbrella points towards the distal direction D. This allows the capture of emboli 15 flowing from the distal direction D towards the proximal direction P by the distal side of the filter 4, i.e within the umbrella.

The filter 4 is penetrable to fluid, so that blood can flow from the distal direction D towards the proximal direction P, through the filter, but particles 15 are captured by the distal side of the filter 4. As such, the filter 4 avoids movement of emboli from the distal direction D towards the proximal direction P by capturing such particles.

In one example, the filter 4 is configured to retain particles 15 having a size of at least 1.2 mm. In particular, any voids or openings in the filter 4, i.e. of the functional element 4 in the deployed configuration, are smaller than about 1.2 mm.

By retracting the first connector 6a in the elongated configuration proximally, the filaments 5, which may be regarded as elongated in the elongated configuration between the first and second connectors 6a, 6b, are folded. Such turnaround points 5c or folds are defined by loops of the filaments 5. The ends or turnaround points 5c represent a smooth and gentle end of the filter 4, which is configured to gently conform and engage with a bodily wall 8. For example, the turnaround points 5c are configured to conform to an inside of a substantially cylindrical bodily wall 8, such as a blood vessel. Loops or turnaround points 5c define returning ends, as opposed to sharp ends/tips.

In the deployed configuration, the first and second connectors 6a, 6b may be located in proximity to each other. Specifically, the first connector may be connected to the second connector 6b. For example, connection may mean that the first connector 6a is in engagement with the second connector 6b. The first connector 6a may be engaged with the second connector 6b to the effect that the second connector 6b restricts movement of the first connector 6a in the distal direction D, i.e. towards the elongated configuration.

The turnaround points 5c in the deployed configuration may be at about half of the length of the filaments 4 in the longitudinal direction in the elongated configuration. Put differently, the distance between the proximal end p and the distal end d-e of the functional element (filter) 4 in the elongated configuration (see FIG. 1) may be about twice the distance between the proximal end p and the distal end d-d of the filter 4 in the deployed configuration (see FIG. 2a).

If the first connector 6a comprises compliant/elastic material, the first connector 6a may deform while passing through the second connector 6b, when the first connector 6a is moved proximally by the actuator 7. After the first connector 6a has passed through the second connector 6b, the first connector 6a may resume its original shape (it may expand), so that the second connector 6b restricts distal movement of the first connector 6a.

The first connector 6a may be smaller than the through hole 6b-1 of the second connector 6b to facilitate passing of the first connector 6a through the second connector 6b.

As indicated in FIG. 2a, the turnaround points 5c which may be in contact with the wall 8 of the body lumen (e. g. vessel wall) are smooth and rounded and avoid damage to the wall 8. FIG. 2a shows a guidewire 11 which passes through the ring or opening 6a-1 of the first connector 6a. The guidewire 11 also passes through a through a hole 6b-1 of the second connector 6b.

FIG. 3 shows a configuration when retracting the first connector 6a from the deployed configuration further proximally. FIG. 3 shows that the actuator 7 comprises a pull wire 7 and a hook 12 for attachment to the first connector 6a.

The distal end d-d of the filter 4 is moved proximally during retraction. This may result in the umbrella-like structure being pulled into the inside of the sheath 3. The umbrella-like structure may help to avoid escape of captured emboli (not shown in FIG. 3) and so that such emboli do not become loose in the vessel 8 during retraction.

During insertion of the catheter 1 of the disclosure into a vessel 8, the actuator 7 and the catheter tube 2 may be fixed relative to each other. The catheter 1 may be inserted through the guiding sheath 3. When the functional element 4 is at its targeted location, the sheath 3 is retracted so that the functional element 4 exits the sheath 3 and assumes its elongated configuration. Then, actuator 7 is retracted. By doing so, the first connector 6a, which may be a distal ring, is retracted towards the second connector 6b, which may be a proximal ring. The functional element 4 unfolds during such deployment and forms the filter 4. By pulling the first connector 6a through the second connector 6b, a final deployed configuration of the filter 4 may be reached. For example, the first connector 6a may be compressed when it is being pulled through the second connector 6b. Pulling the first connector 6a through the second connector 6b provides fixation of the first and second connectors 6a, 6b relative to each other and, hence, ensures that the filter 4 is maintained in the deployed configuration during use.

For retraction of the filter 4, i.e. for removal from the vessel wall 8, the first connector 6a may be pulled proximally by the actuator 7, as described above. Alternatively, the second connector 6b may be retracted, or both first and second connector 6a, 6b may be pulled by means of the actuator 7. The filter 4 may be withdrawn inside (another) guiding sheath 3, as this will help to ensure that the captured emboli do not escape during removal of the filter 4.

As reflected in FIG. 2b, the filaments 5 forming the functional element 4 may have an approximately equal angular distribution, forming, in a cross section perpendicular to the proximal/distal direction, a circular opening 13. The size of such circle may depend on the distance between the first and second connectors 6a, 6b in the elongated configuration, i.e. a length of the filaments 4 and also on the diameter of the body lumen 8.

The catheter 1 may be a vascular catheter for use in blood vessels.

A catheter system may comprise the catheter 1 and the sheath 3.

### REFERENCE SIGNS

1 catheter
2 catheter tube
3 sheath
4 functional element (filter)
5 filaments
5a first end
5b second end
5c turn-around point
6a first connector
6a-1 opening in first connector
6b second connector
6b-1 opening in second connector
7 actuator (pull wire)
8 bodily (vessel) wall
11 guidewire
12 hook
13 opening of filter
14 virtual outer shell in the elongated configuration
15 particles (emboli)
P proximal direction
D distal direction
d-e distal end of functional element in elongated configuration
d-d distal end of functional element in deployed configuration
p proximal end of functional element

1. A catheter, comprising: a functional element having at least a deployed configuration and an elongated configuration, the functional element comprising filaments and first and second connectors, the first connector connected to a first end of the filaments, and the second connector connected to a second end of the filaments, the second connector (6b) including a through-hole (6b-1) configured for the first connector (6a) to pass through, wherein
in the elongated configuration, the first connector is at a distal end of the functional element and the second connector is at a proximal end of the functional element, and
in the deployed configuration, the functional element is configured to act as a filter penetrable to fluid, the filaments forming turn-around points forming a distal end of the filter and configured to conform to a substantially cylindrical bodily wall of a bodily lumen, wherein, in the deployed configuration, the first connector is proximal to the turn-around points, the catheter further comprising an actuator configured to move the first connector proximally and to thereby facilitate transformation of the functional element from the elongated configuration into the deployed configuration of the functional element.

2. The catheter of claim 1, wherein the first connector is located proximal to the second connector in the deployed configuration.

3. The catheter of claim 1, wherein the actuator comprises a pull-wire configured to pull the first connector in the proximal direction.

4. The catheter of claim 1, wherein the second connector is ring-shaped.

5. The catheter of claim 1, wherein the first connector is configured to be smaller than the through-hole of the second connector.

6. The catheter of claim 1, wherein the first connector has a central opening for a guidewire (11) to pass through and/or the central opening is ring-shaped.

7. The catheter of claim 1, wherein the filaments form a mesh.

8. The catheter of claim 1, wherein in deployed configuration, the filaments from an umbrella shape, the open side of the umbrella facing in the distal direction.

9. The catheter of claim 1, wherein in deployed configuration, the first and second connectors are located in proximity, optionally the first connector is connected to the second connector.

10. The catheter of claim 9, wherein the first connector comprises compliant material such that the first connector is configured to deform to pass through the second connector when moved proximally by the actuator and configured to resume its original shape when proximal to the second connector.

11. The catheter of claim 1, wherein in the elongated configuration, an outer shape of the entirety of the filaments is at least partially cylindrical.

12. The catheter of claim 1, wherein in the deployed configuration, the turn-around points are configured to conform to an inner surface of a blood vessel.

13. The catheter of claim 1, wherein the second connector is arranged distal to or at the distal end of a tube of the catheter.

14. A method of using a catheter, the method comprising:
providing the catheter the catheter including a functional element having at least a deployed configuration and an elongated configuration, the functional element comprising filaments and first and second connectors, the first connector connected to a first end of the filaments, and the second connector connected to a second end of the filaments, the second connector (6b) including a through-hole (6b-1) configured for the first connector (6a) to pass through, wherein
in the elongated configuration, the first connector is at a distal end of the functional element and the second connector is at a proximal end of the functional element, and
in the deployed configuration, the functional element is configured to act as a filter penetrable to fluid, the filaments forming turn-around points forming a distal end of the filter, wherein the first connector is proximal to the turn-around points,
the catheter further comprising an actuator configured to move the first connector proximally and to thereby facilitate transformation of the functional from the elongated configuration into the deployed configuration of the functional element,
retracting the actuator and transforming the functional element from the elongated configuration to the deployed configuration, wherein the filter conforms to a substantially cylindrical vessel wall at the turnaround points, and
capturing emboli from blood flow by way of the filter.

15. The method of claim 14, further comprising:
separating the functional element in the deployed configuration from the actuator and retracting the actuator.

16. The method of claim 14, further comprising:
introducing the actuator and connecting the actuator to the filter.

17. The method of claim 14, further comprising:
transporting the captured emboli proximally by retracting the actuator.

18. A method of retaining emboli, the method comprising:
providing a catheter having a functional element having at least a deployed configuration and an elongated configuration, the functional element comprising filaments and first and second connectors, the first connector connected to a first end of the filaments, and the second connector connected to a second end of the filaments, the second connector (6b) including a through-hole (6b-1) configured for the first connector (6a) to pass through, wherein
in the elongated configuration, the first connector is at a distal end of the functional element and the second connector is at a proximal end of the functional element, and
in the deployed configuration, the functional element is configured to act as a filter penetrable to fluid, the filaments forming turn-around points forming a distal end of the filter, wherein the first connector is proximal to the turn-around points,
the catheter further comprising an actuator configured to move the first connector proximally and to thereby facilitate transformation of the functional element from the elongated configuration into the deployed configuration of the functional element;
retracting the actuator and transforming the functional element from the elongated configuration to the deployed configuration, wherein the filter conforms to a substantially cylindrical vessel wall at the turnaround points, and
capturing emboli from blood flow by way of the filter.

19. The method of claim 18, further comprising:—separating the functional element in the deployed configuration from the actuator and retracting the actuator.

20. The method of claim 18, further comprising: —introducing the actuator connecting the actuator to the filter.

21. The method of claim 20, further comprising:—transporting the captured emboli proximally by retracting the actuator.
